(19) United States
(12) Patent Application Publication
(10) Pub. No.: US 2022/0208321 A1
(43) Pub. Date: Jun. 30, 2022

(54) HEALTH RECORD SYSTEM

(71) Applicant: VISUAL TERMINOLOGY INC., Busan (KR)

(72) Inventor: Byung Kwon CHOI, Busan (KR)

(73) Assignee: VISUAL TERMINOLOGY INC., Busan (KR)

(21) Appl. No.: 17/607,276

(22) PCT Filed: Apr. 6, 2020
(86) PCT No.: PCT/KR2020/004612

§ 371 Date: Oct. 28, 2021

(30) Foreign Application Priority Data
Jun. 24, 2019 (KR) 10-2019-0074956

(51) Int. Cl. G16H 10/60; G16H 30/40; G16H 50/50

(52) U.S. Cl. CPC G16H 10/60; G16H 50/50; G16H 30/40

(57) ABSTRACT

Disclosed is a health record system which includes a reception module configured to receive medical data including medical information generated in a medical institution; a data conversion module configured to extract text-type data from the medical data; a visualization module configured to generate image-type data by using the text-type data extracted by the data conversion module; a display module configured to display the image-type data to a user; and a storage-transmission module configured to store and transmit the data generated by the reception module, the data conversion module, and the visualization module.

### CROSS-REFERENCE TO PRIOR APPLICATIONS

This application is a National Stage Patent Application of PCT International Patent Application No. PCT/KR2020/004612 (filed on Apr. 6, 2020) under 35 U.S.C. § 371, which claims priority to Korean Patent Application No. 10-2019-0074956 (filed on Jun. 24, 2019), which are all hereby incorporated by reference in their entirety.

### BACKGROUND

The present invention relates to a health record system that simply and accurately organizes medical information existing across multiple hospitals in a place and enables a patient to transmit symptom information of the patient to a medical institution (H) even without knowing medical terminology.

A field of recording health has made much progress through the grafting of IT technology, and one of them is an electronic medical record system that processes charts that have existed on paper as electronic information. The electronic medical record system has contributed greatly to the efficient operation of the hospital. For example, medical staffs may inquire and record patient's information when opening only an electronic medical record system anywhere in the hospital, and nurses, physical therapists, etc. may do different tasks in different places at the same time. As a result, the work efficiency has been improved, a space in which the information has been stored as the paper chart has been saved, and an unnecessary logistical work, which had to carry the charts, and the like have disappeared. However, even in these information, from a personal aspect, there are multiple hospitals that provide treatment. As a result, there is a movement to collect and manage a history in which the patient has been treated previously across multiple hospitals at once. In many other countries including the United States and Korea, many efforts have been made to establish a platform that allows individual health information to be collected and be easily accessible by parties through the hospital information exchange. This has been shown in the form of Blue Button in the US, My Health Record in Australia, Google Health in Google, and Health Vault in Microsoft. Such a platform may do not just inquire information, but also enable individuals to directly manage their health and may perform a function of providing health management services before disease occurs.

Also, there is a difference from an electronic medical record (EMR) or a hospital information system (HIS) used in hospitals in that an individual is the owner of information. This individual unit of information in the current technology is information having a complex structure and has a data structure that is not easy for an individual to manage. For example, from a patient's point of view, medical terminology is unfamiliar, making it difficult to understand information expressed in medical terminology. This is because, for example, the patient is not a medical expert who knows an anatomical name or a disease name well. In addition, a difference in language for the same medical information may also be a problem.

Organization of medical data is also a problem. Basically, in the medical data, complex medical events, such as the occurrence of symptoms, outpatient care, hospitalization, conduction of various tests, confirmation of disease, administration, and changes in disease conditions, may occur at similar times in multiple hospitals. It is difficult to organize these data well with current medical data. If there are dozens of medical records when a patient undergoes appendectomy and is discharged and these medical records are collected over a lifetime, a set of tens to hundreds of thousands of texts makes it difficult to easily identify the patient's condition.

In addition, since the disease is expressed only in medical terminology in terms of an individual who is not a medical staff, it is difficult to understand the disease, and thus there is a barrier in that it is difficult for a general public to use personal health records.

On the other hand, there is a problem even when a patient wants to record symptoms of the patient and transmit the recorded symptoms to the hospital. The patient cannot express painful spots of the patient in medical terminology, and in particular, information prepared by the patient in a narrative description without knowing standard medical terminology is unclear in meaning and incomprehensible to the medical staff, and is difficult to be structured in a material form, so that it is difficult to use the information as professional medical information in hospitals.

In the present invention, complex medical events occurring in multiple hospitals are organized in image-type data (310) using figures by compensating for character (text-type) information which has been a main form of existing medical information. Therefore, there is invented a new health record system capable of being easy for patients to understand data, organizing and viewing medical data well. and easily expressing data prepared in multiple hospitals with different languages.

### SUMMARY

The present invention is derived to solve the problems and an object of the present invention is to provide a health record system provided to simply and accurately organize personal medical information existing across multiple hospitals in a place by using image-type data (310).

Further, an object of the present invention is to provide generation of medical data (210) and a system thereof provided to enable a patient to transmit accurate information to a medical institution (H) by displaying painful spots of the patient on the figure even while the patient does not accurately know medical terminology.

The technical objects of the present invention are not limited to the aforementioned technical objects, and other technical objects, which are not mentioned above, will be apparently appreciated by a person having ordinary skill in the art from the following description.

A health record system according to the present invention comprises: a reception module (100) configured to receive medical data (210) including medical information generated in a medical institution (H);

a data conversion module (200) configured to extract text-type data (220) from the medical data (210);

a visualization module (300) configured to generate image-type data (310) by using the text-type data (220) extracted by the data conversion module (200); a display module (400) configured to display the image-type data (310) to a user; and

a storage-transmission module (500) configured to store and transmit the data generated by the reception module (100), the data conversion module (200), and the visualization module (300).

The image-type data (310) generated by the visualization module (300) may be a predetermined 2D or 3D model.

The data conversion module (200) may collect the medical data (210) by receiving materials from any one or more of a portable file, a hospital, a cloud server, and a personal device.

The visualization module (300) may express the name of a disease, the severity of the disease, the chronicity, the degree of malignancy, various test results, functional test results, and data results extracted from a machine as any one or more of colors, brightness or transparency, patterns, and textures (320) of the visualization data.

The visualization module (300) may use an image extracted from the medical image or an anatomical pathology photograph as the texture (320).

By the technical solution, according to the present invention, it is possible to simply and accurately organize personal medical information existing across several hospitals in a place. Such medical information may be visualized and displayed on a personal device for individuals without professional medical knowledge.

Further, it is possible to easily transmit symptom information to the medical institution (H) even when the patient does not clearly know the medical terminology in which the symptom information of the patient is recorded. Particularly, it is possible to eliminate linguistic barriers by managing various information with different languages as the image-type data (310).

Further, according to the present invention, it is possible to provide a health record system capable of generating 2D or 3D image-type data from medical data (210) which is generated in each hospital to convert the medical data (210) into image-type data.

Further, according to the present invention, it is possible to provide a health record system capable of standardizing text-type data based on the image-type data (310), wherein the text-type data are generated in each hospital that has different text lengths and types of data while having ambiguous meanings.

Further, it is possible to provide a health record system capable of having richer meanings by converting medical information recorded as the text-type data into the image-type data.

In addition, the present invention may be used for chronic disease management, blood pressure nutrition, exercise management, etc. of each patient.

In addition, the present invention enables collective management of diseases nationally by converting individual disease data into standardized image-type data (310).

In addition, by using the system of the present invention, the user's disease data may be remotely transmitted to other hospitals or personal devices to be efficiently used for customized consultation and treatment.

In addition, by using the system of the present invention, it is possible to provide a national-level infrastructure capable of not only improving medical quality and efficiency but also contributing to the development of medical-related industries and economic development through this.

### BRIEF DESCRIPTION OF THE DRAWINGS

FIG. 1 is a schematic diagram illustrating a configuration of a health record system of the present invention.

FIG. 2 is a schematic diagram illustrating an embodiment for the use of the health record system.

FIG. 3 is a schematic diagram illustrating an embodiment of allowing a patient to record symptoms of the patient and to transmit information to hospitals.

FIG. 4 is a schematic diagram schematically illustrating a configuration of converting medical data (210) into a 2D medical information model (312) in a configuration of a data conversion module (200).

FIG. 5 is a schematic diagram schematically illustrating a configuration of converting the medical data (210) into a 3D medical information model (312) in the configuration of the data conversion module (200).

FIG. 6 is a schematic diagram schematically illustrating a configuration of adding the medical information model (312) to a basic model (311) in the configuration of the data conversion module (200).

FIG. 7 is another schematic diagram schematically illustrating a configuration of adding the medical information model (312) to a 2D basic model (311) in the configuration of the data conversion module (200).

FIG. 8 is an embodiment illustrating a round pattern (321), a thin diagonal pattern (322), a thick diagonal pattern (323), and a dotted diagonal pattern (324) in a configuration of a visualization module (300).

FIG. 9 is a photograph illustrating an embodiment capable of using images extracted from a medical image, anatomical pathology findings, skin disease photos, etc. as a texture (320).

FIG. 10 illustrates an embodiment of the image extracted from the medical image and is a diagram illustrating a case where a part of a CT image is taken to generate a texture (320) for a medical information model (312) of a patient with brain hemorrhage.

FIG. 11 is a diagram showing using one or more layers according to characteristics of the medical data (210) to further expand information expression of the visualization module (300).

FIG. 12 is a diagram for describing the medical information model (312) that further expresses diseases or symptoms which are not able to be anatomically expressed inside and outside the body in the basic model (311).

FIG. 13 is an embodiment illustrating a method of extracting data by the data conversion module (200) when the text-type data (220) for generating the image-type data (310) is stored in the medical data (210) as a separate item.

FIG. 14 is an embodiment illustrating a method of extracting data by the data conversion module (200) when the text-type data (220) for generating the image-type data (310) is stored in the medical data (210) for each “diagnosis name”.

FIG. 15 is an embodiment illustrating a method of extracting data as the text-type data (220) by the data conversion module (200) when the text-type data (220) for generating the image-type data (310) is free-text data which is not organized into separate items or unstandardized strings or binary large object (BLOB)-type data in a database.

FIG. 16 is an embodiment illustrating a form of visualizing data in one or more basic models (311) by the visualization module (300).

FIG. 17 is an embodiment showing a means for managing a plurality of patients in a hospital or a country.

FIG. 18 is an embodiment illustrating a display module (400) that allows a user to execute another application by processing a GUI event.

### DETAILED DESCRIPTION

Terms used in the present specification will be described in brief and the present invention will be described in detail.

Terms used in the present invention adopt general terms which are currently widely used as possible by considering functions in the present invention, but the terms may vary depending on an intention of those skilled in the art, a precedent, emergence of new technology, etc. Accordingly, the terms used in the present invention should be defined based on not just a name of the term but a meaning of the term and contents throughout the present invention.

Throughout the specification, when any part “comprises” any component, the part may further include other components instead of excluding other components unless specifically stated otherwise.

An embodiment of the present invention will be described more fully hereinafter with reference to the accompanying drawings so as to be easily implemented by those skilled in the art. However, the present invention may be embodied in many different forms and is not limited to embodiments described herein.

Specific matters including problems to be solved for the present invention, solutions of the problems, and the effects of the invention for the present invention are included in embodiments and drawings to be described below. Advantages and features of the present invention, and methods for accomplishing the same will be more clearly understood from embodiments described in detail below with reference to the accompanying drawings.

Hereinafter, the present invention will be described in more detail with reference to the accompanying drawings.

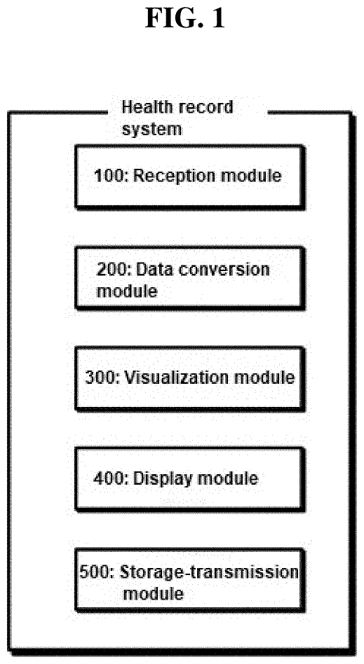

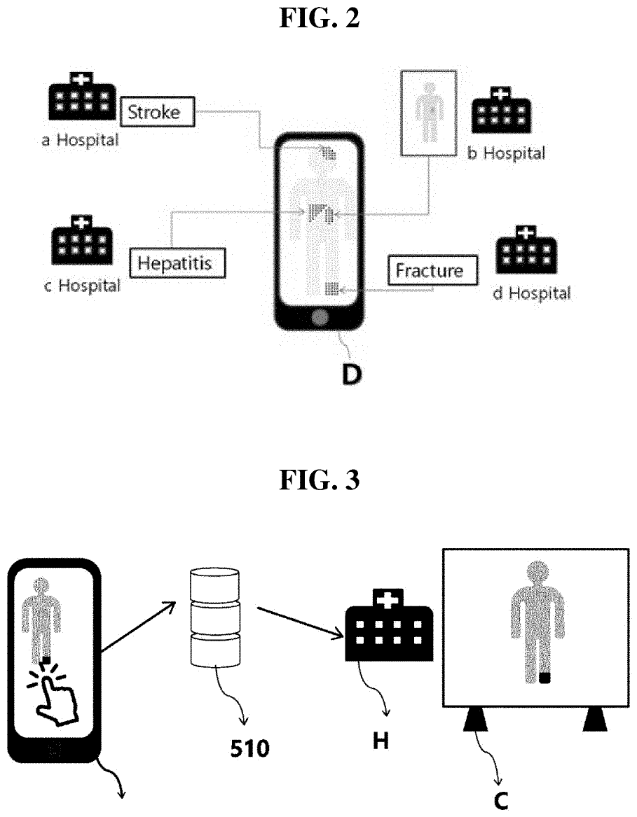

As illustrated in FIG. 1, a health record system of the present invention is constituted by a reception module 100, a data conversion module 200, a visualization module 300, a display module 400, and a storage-transmission module 500. As illustrated in FIG. 2, the personal health record generated by the health record system of the present invention is transmitted to a terminal D by the storage-transmission module 500 and patient's information required by the patient or a medical institution (H) is displayed on a screen of the terminal D. Here, the terminal D may be a computer used by a medical staff or a personal device used by a patient personally. For example, the patient may inquire patient's personal health record from a patient's smart phone.

First, the reception module 100 receives medical data 210 including medical information generated in the medical institution H. The reception module 100 may be configured to have at least one of a communication device, software, or a server capable of collecting personal medical information which has been scattered in each hospital.

Next, the data conversion module 200 extracts text-type data 220 for creating image-type data 310 from the medical data 210. A subject to be extracted may be a clinical document received via USB or e-mail, as well as a hospital, or electronic medical record system materials stored in the hospital. These materials may be stored in document forms or stored in a database as materials. Even when the patient visits as an outpatient or is hospitalized several times, the data conversion module 200 serves to extract data of a required form for creating the image-type data 310 from the text-type data 220. Further, the data conversion module 200 may extract the data from materials received from other hospitals or even from materials stored in a server outside the hospital, materials stored in a personal device, or materials received from various medical devices. Meanwhile, the image-type data 310 refers to digitized data visually representing medical information such as a person, an organ, and a disease. These image data 320 may include Joint Photographic Experts Group (Jpeg), Graphics Interchange Format (GIF) Scalable Vector Graphic (SVG), Tag Image File Format (TIFF), Portable Network Graphics (PNG), and Standard Triangulated Language (STL), OBJ, Filmbox (FBX), Collabarative Design Activity (COLLADA), etc., and may include 2D or 3D data which are not mentioned herein. The image-type data 320 may be constituted by one or several combinations, and a change in time may be represented by a combination of multiple image-type data 320.

The text-type data 220 means text-type data included in the medical data, such as disease names or diagnosis names, symptoms, blood test results, reading papers, surgical names, nursing records, and nursing measures, as data acquired from the medical data 210 represented as clinical records, electronic medical records, progress recodes, discharge summaries, medical terminologies, or other many text types or number types.

The text-type data 220 is not limited to a diagnosis name, and the text-type data 220 may include data defined in anatomical sites, procedure names, measured blood pressure values, and the activity of a patient of a massage medical staff or a medical assistant, or various text-type materials indicating patient's conditions such as “serious”, “light”, “large”, and “small”. For example, the text-type data 220 may be characters expressing various languages such as Korean or English, such as “fatty liver”, “ankle pain”, and “heart failure”, or standardized data, standardized medical terminology, or medical terminology codes, such as “K76.0”, “61515”, “N05”, and “M51.0”, which are numbers or combinations of characters and numbers. The standardized medical terminology code refers to data in which medical concepts are defined in SNOMED-CT, ICD-9, ICD-10, ICD-11, LOINC, CPT, ATC, RxNorm, ICNP, NMDS, and the like. In addition, a test result of a hemoglobin level of 10.3 gram/deciliter may be data expressed by numbers.

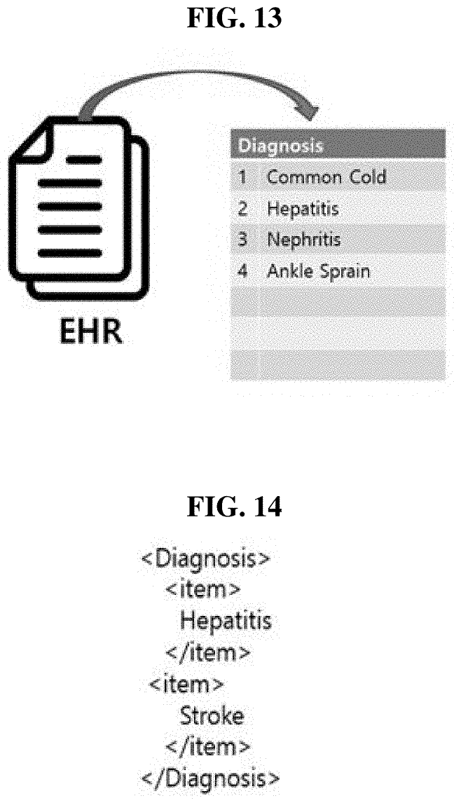

FIGS. 13 and 14 are an embodiment illustrating that data required for extracting the text-type data 220, such as a medical record document, are stored in the electronic medical record system as separate items. For example, a data set defined as a text-type “personal health record” may exist as a separate system. In addition, in the data stored in the form of a document, as illustrated in FIG. 14, data in the form of JSON or XML may be classified for each diagnosis name, each surgical name, or each symptom, and at this time, the required items may be read and taken. In this case, the text-type data 220 may be extracted by accessing the database and reading only the required items. Meanwhile, the type of the data may also include materials for creating the image-type data 310 rather than the existing text-type data. For example, image attributes, such as a unique number of the basic model, an identification number (ID) of the medical information model, a location of the medical information model inside the basic model, a texture type, brightness, transparency, and colors, may be included. In addition, a separate data set for managing data related to such image-type data may be defined in the “personal health record”.

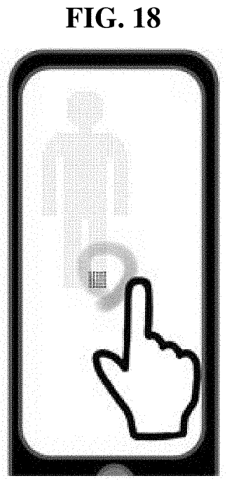

In addition, when the text-type data 220 is free statement data that is not organized into separate items, unstandardized strings, or data in the form of binary large object (BLOB) data in a database, as illustrated in FIG. 18, a list of the text-type data 220 may be specified for the required items, and necessary values may be extracted. The data conversion module 200 is configured to collect information from data scattered in various hospitals and distributed to external servers.

Next, the visualization module 300 generates the image-type data 310 using the text-type data 220 extracted from the data conversion module 200. More specifically, the acquired text-type data 220 is converted into image-type data 310.

At this time, the image-type data 310 is a predetermined 3D model, and may be generated by combining one or more medical information models 312 with a basic model 311 which is a 3D model.

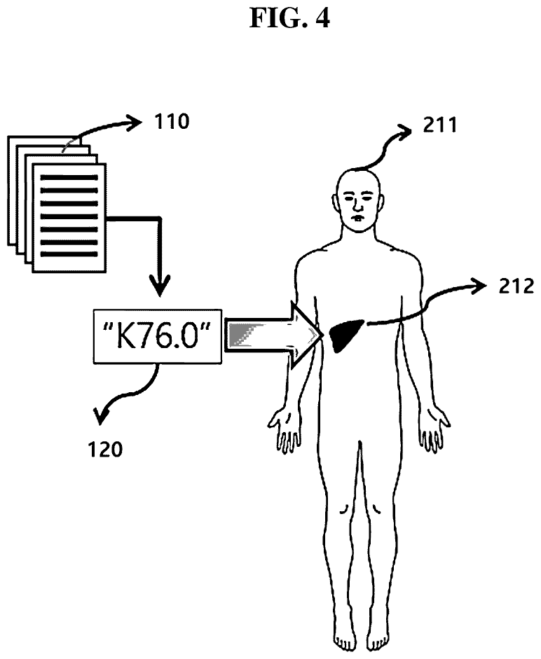

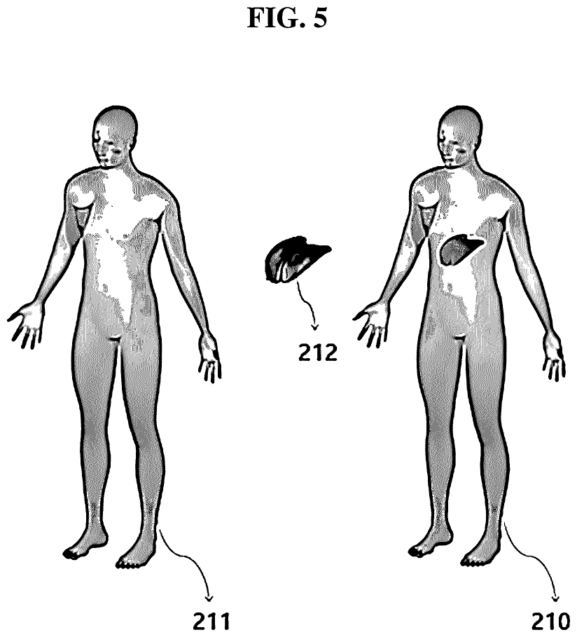

As illustrated in FIGS. 4 and 5, the information representing the medical data 210 is expressed by the image-type data 310. The image-type data 310 includes the medical information model 312 representing the text-type data 220 acquired from the medical data 210 as an image in the basic model 311 which is an image including a certain part of the human body. The medical information model 312 may be expressed in more detail by dots, lines, areas, volumes, or various shapes or combinations thereof, and may be expressed as a 2D model as well as a 3D model. In this process, when predetermined text-type data 220 is input under certain conditions, the image-type data 310 may be automatically generated, and may be generated automatically by using any function of using the text-type data 220 as an input factor or one or more machine learning algorithms such as a generative model.

As illustrated in FIG. 4, the basic model 311 may include the plurality of medical information models 312 to provide the image-type data 310, and a plurality of medical information models 312 which are pre-made and stored may also be used as a medical information model 312 of a certain patient by referring to the text-type data 220 extracted from the medical data 210.

The basic model 311 may also be an empty space in which no picture is drawn, and at this time, the visualization module 300 may express the medical information model 312 in the empty space. In addition, the image-type data 310 may also represent a whole body or only some systems of the human body, such as a digestive system or a respiratory system, or may represent only a certain area of the body, such as a left leg or a head.

The medical information model 312 may be the shape of entire organ or part of an organ. For example, in the case of a tumor occurring from the liver, shape of the entire liver organ or a portion of liver where the tumor occurs (e.g., posterior lateral segment) may be expressed as the medical information model 312. Alternatively, the shape of tumor mass may be expressed as the medical information model 312 regardless of the anatomical fraction of the liver.

The medical information model may be a detailed model that follows anatomical features, but may also be a simple figure such as a curve, a circle, a straight line, a quadrangle, a sphere, and a cube obtained by hand drawing.

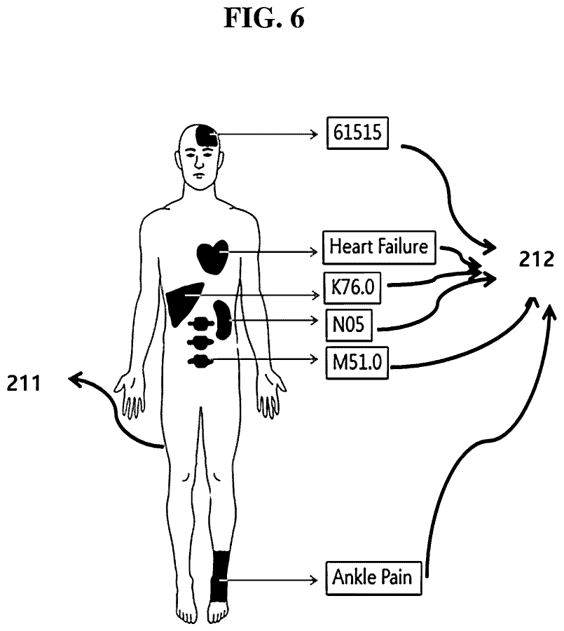

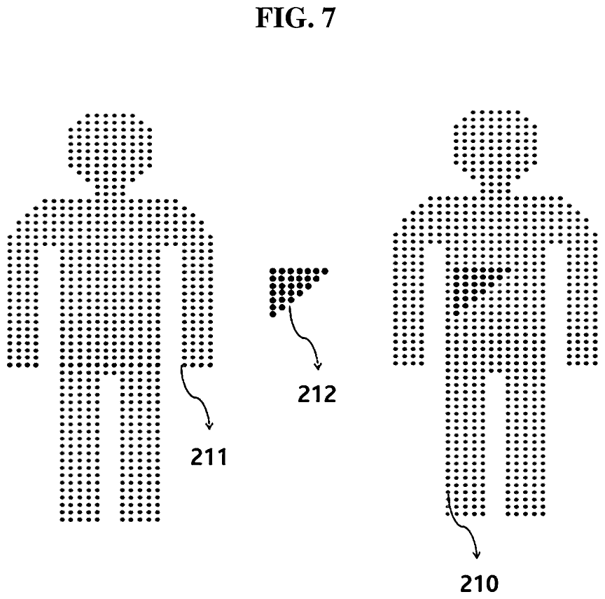

In addition, the medical information model 312 is automatically selected by the extracted text-type data 220 and may be automatically combined without human intervention, however, sometimes it is not extracted from the previously-created medical data 210. It can be added directly from the beginning or drawn by the user. In the medical information model 312, a patient or a medical staff may directly draw a painful spot, or express a site with a spot, an itchy site, a site where a blood pressure is checked, a site where a nail is cut, and a site to be injected. For example, when the patient expresses the site to be injected, the medical staff may record text-type materials in the medical data 210 and then directly draw the basic model 311 in the background without converting the text-type materials into the image-type data 310. Alternatively, when the patient has a fatty liver, the medical staff may directly draw fatty liver against the backdrop of basic model 211 without writing the medical data 210. Alternatively, as illustrated in FIG. 6, the medical staff or the patient may select one of the medical information models 312 pre-made in advance to express a disease condition of a certain patient.

The visualization module 300 may vary the color, brightness, or transparency of the image according to the name of the disease, the severity of the disease, the chronicity, and the degree of malignancy. That is, the visualization module 300 may express the characteristics of the medical data 210 as attribute values of the image-type data 310. For example, the visualization module 300 may determine the color of the image by selecting or combining any one or more of red, green, and blue colors. As an example, when a patient with tibialis anterior muscle paralysis is expressed, if the muscle strength of the corresponding muscle is 20% of the normal, an R value representing the red color among RGB channels which is the color expressing the muscle strength may represent the muscle strength with 20% of the maximum value, and if the maximum value of the red channel is 255, the R value may be expressed as 255*0.2=50.5. On the other hand, the function of the kidney may be checked by an estimated glomerular filtration rate (eGFR) as one of the blood tests, and may be represented as 255 if the eGFR is 100 and 255/2 which is half of 255 if the eGFR is 50 in conjunction with a G value representing the green value to the eGFR value. If the eGFR is 0, the G value may be represented as 0. That is, the patient's condition may be represented by changing and expressing the attributes of the image by a function of using a result value of the blood test as a factor. As such, the color may be defined as a value determined by a function of using clinical data as a factor.

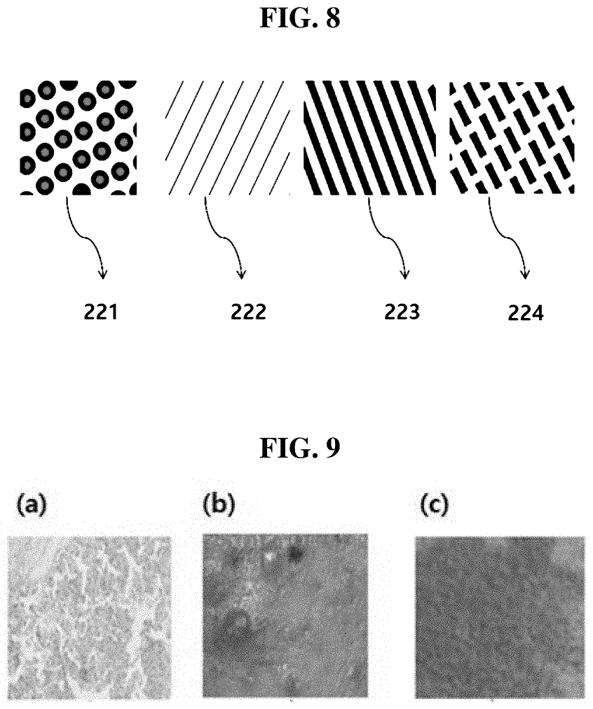

As illustrated in FIG. 8, in order to enhance visibility so that a person may easily recognize required information, the image-type data 310 may be completed in the medical information model 312 by applying a texture 320 to the basic model 311 in addition to general image attributes such as color, brightness, and transparency.

In addition, the visualization module 300 may express the texture 220 in the medical information model 312 according to a name of the disease, a medical terminology code, the chronicity, severity, and malignancy. For example, as illustrated in FIG. 8, the stenosis may be expressed by a round pattern 321, the squamous cell carcinoma may be expressed by a thin diagonal pattern 322, the hemangioma may be expressed by a thick diagonal pattern 323, and the paralysis may be expressed by a dotted diagonal pattern 324. The patterns presented herein represent a few of examples of the texture 320, and the texture 320 is not limited thereto and may be prepared by using a man-made figure, an icon representing a disease, or the like.

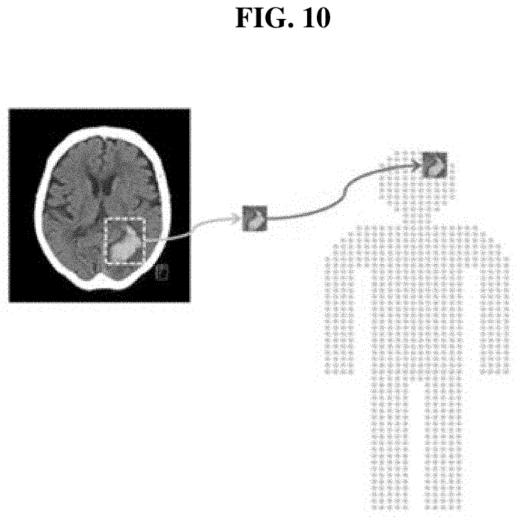

In addition, the visualization module 300 may use an image extracted from the medical image, a photograph or image showing an anatomical pathology finding, and an image to be photographed or extracted such as a skin disease photograph, as the texture 320. That is, all medical images may be applied to the medical information model 312. For example, in FIG. 9A, microscopic tissue findings may be used as the texture 320, and typical pathological findings that may well express the patient's condition or a photograph of the corresponding patient may be taken directly. FIG. 9B is an image of photographing a skin lesion, and the image may be used as the texture 320 in a corresponding region and may also be a photograph of directly photographing a patient's skin. FIG. 9C is a part of an image photographed by an MRI. In addition, FIG. 10 illustrates a case in which a part of the CT image is taken to make the texture 320 of the medical information model 312 of a patient with brain hemorrhage. Accordingly, it is possible to reduce the trouble to check the medical image again to determine the patient's condition, and to transmit more accurate information to the medical user.

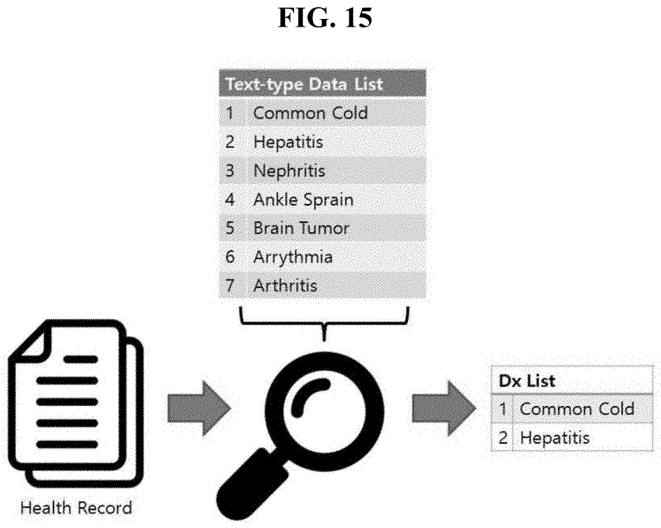

The visualization module 300 may further include the medical information model 312 that further expresses patient's diseases or symptoms which are not able to be anatomically expressed inside and outside the body shown in the basic model 311. Based on the basic model 311 expressing the human body, there may be more medical information that cannot be expressed by the medical information model 312. For example, the medical information is high blood pressure and diabetes. Of course, in the case of diabetes, the malfunction of the pancreas may be the cause, but when a relationship with the pancreas cannot be confirmed, it may be difficult to express information due to this pancrease condition. FIG. 15 illustrates an embodiment of the medical information model 312 that additionally shows a model of diabetes and hypertension outside the body to compensate for this. These models may also be used as a concept to express abstract conditions such as “anxiety” and “critical vital signs”.

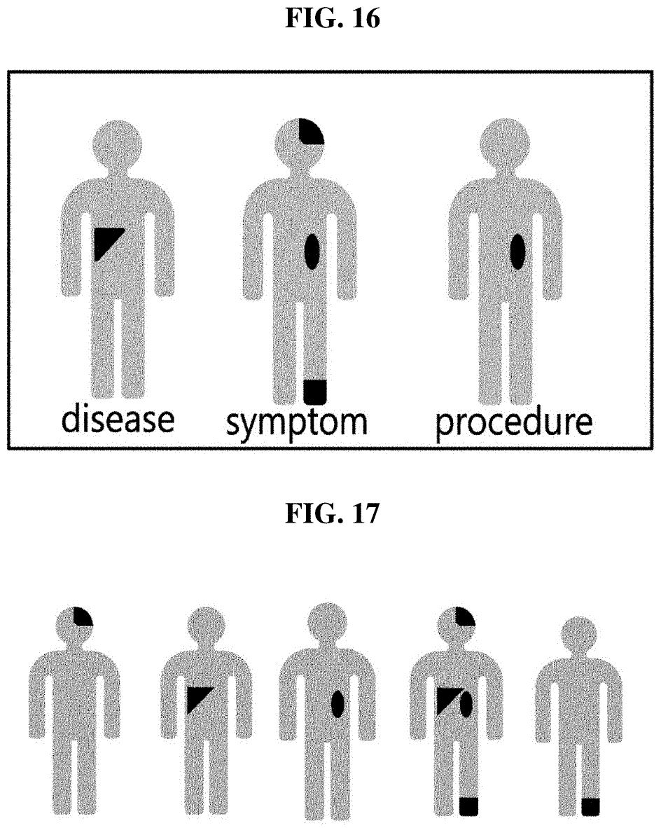

FIG. 16 illustrates a form of visualizing data on categorized basic models 311 by the visualization module 300, and illustrates an example of visualizing information classified by disease, symptom, and procedure in each of the basic models 311 as the medical information model 312. In a manner in which the data is separately recorded according to the classification of the text-type data 220 in the plurality of basic models 311, in this case, there are advantages of managing the medical data 210 by type as needed.

Next, the display module 400 serves to show the image-type data 310 to the user. The display module 400 has a function of displaying the image-type data 310 to the user through a screen of a user's application or a computer. The display device including the display module 400 is provided with a predetermined graphical user interface (GUI) and also performs a function of receiving user's input information. For example, the display device may display additional information describing specific diseases on the screen while showing the image-type data viewed by the patient. The display device may display at least one of a simple disease name or an occurring time point, that is, the date, a type of drug to be administered, and chronicity.

The display module 400 can activate other programs by using the GUI. Here, the GUI may be an event such as a finger touch, a mouse event click, a wheel, a mouse leaving GUI focusing keyboard input, a sound recognition, a device operation, etc., and may execute other applications in the system rather than the health record system by using these events. For example, the patient may execute another application when clicking a part of the basic model or clicking a part of the medical information model. At this time, the application may show a visit history of hospitals that the patient has visited due to an abnormality in the corresponding body part. Alternatively, the application may be a product inquiry screen that shows a list of medical device shopping malls or products that are required when the corresponding area is ill. The type of the application or the data to be shown may be determined by a function of using location information of the GUI event executed by the user as a factor. For example, when the patient clicks a foot on the screen or displays an area, the patient performs a search by receiving the clicked location and the size of the area, and then may show an athlete's foot related to the corresponding location or a list of shopping malls related to shoes. Alternatively, if the patient displays the heart in a circle, a homepage of a hospital for treating the heart disease may be searched and displayed.

Meanwhile, the time point of the disease condition may be converted by using the GUI event. The time point of the visualized image may be converted by using a button, a slider, or a scroll. Based on the example, by using a swipe, a state in which nephritis occurs after a hospital B is visited is shown and then may move to a time point when the nephritis occurs previously via a button or a swipe event. Alternatively, by other forward buttons or swipe events in different directions, a state after hepatitis has occurred after a hospital C is visited may be displayed. Alternatively, the state may freely move to a required time point in a short time by using a scroll bar.

Meanwhile, in the medical data, complex medical events, such as the occurrence of symptoms, outpatient care, hospitalization, conduction of various tests, confirmation of disease, administration, and changes in disease conditions, may occur simultaneously in multiple hospitals. The visualization module 300 may create the image-type data 310 reflecting various medical events according to the variation of time to provide the image-type data 310 to a personal health record storage. That is, multiple image-type data 310 generated by the visualization module 300 may be generated as needed. For example, first data may be generated from information on hospitals visited at the age of 20, second data may be generated from information on hospitals visited at the age of 30, and third data may be generated from information on hospitals visited at the age of 40, so that a total of three data may be generated. As described above, the image-type data 310 reflecting the passage of time may also be provided by making temporal data or videos using the plurality of image-type data 310 having different time points. That is, the visualization module 300 may confirm a trend of the data according to a change in time point by combining data created at various time points.

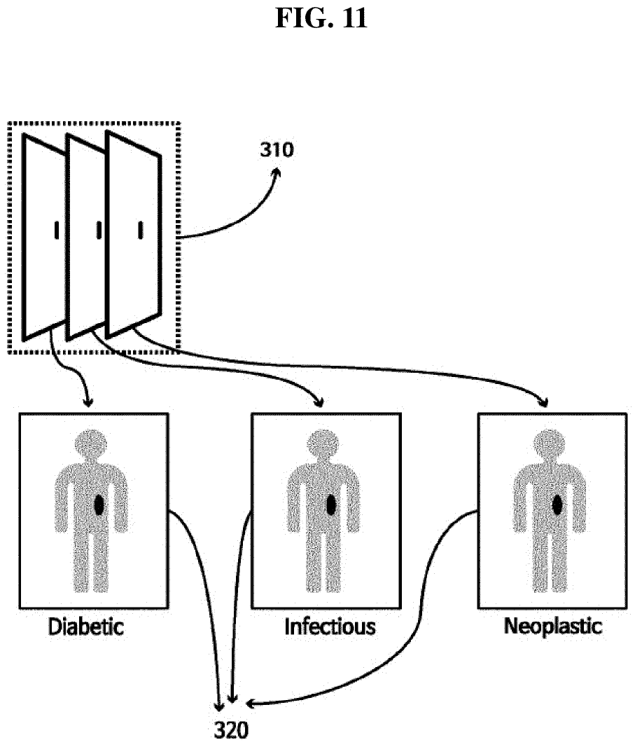

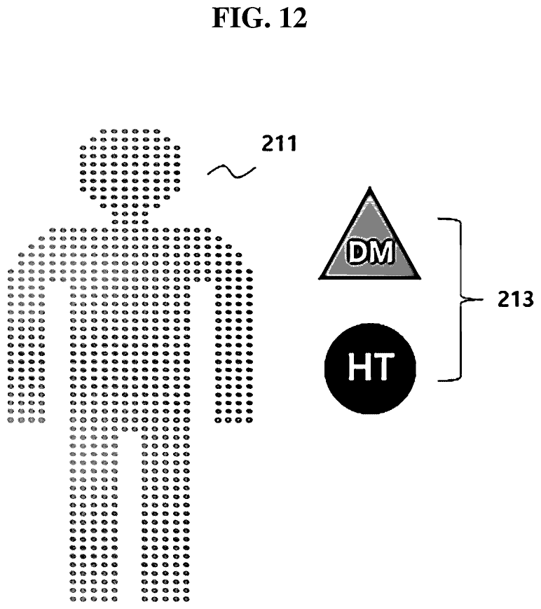

The visualization module 300 is characterized to use one or more layers according to the characteristics of the medical data 210 to further expand expression of information. When there are multiple diseases in the kidney, the disease conditions may be expressed using multiple layers. As illustrated in FIG. 11, diabetic kidney disease, infectious kidney disease, and neoplastic kidney disease may be simultaneously expressed and represented.

Next, the storage-transmission module 500 stores and transmits the data generated by the reception module 100, the data conversion module 200, and the visualization module 300. The storage-transmission module 500 stores the data generated in each module through a remote storage 510 or a personal terminal D, and may transmit the image-type data 310 in an electronic medical record system C of the medical institution H.

Hereinafter, the health record system of the present invention will be described in more detail through an embodiment.

FIG. 2 is a schematic diagram illustrating an embodiment of the health record system. If there is an event in which a patient suffered from the stroke and was treated at a hospital a, while the image-type data 310 of the patient expressing the condition before the stroke occurred through the health record system of the present invention is stored in the patient's personal terminal D, the visualization module 300 adds current disease information of the patient with stroke, or replaces the data with the image-type data 300 added with the stroke.

Over time, the patient develops nephritis again and is treated at a hospital b. A patient who has hospitalized and treated at the hospital b will be treated as an outpatient. For the hospitalization event at this time, the medical information model 312 transmitted from the electronic medical record system C of the hospital b is transmitted and displayed to the patient's personal terminal D. The stroke treated in the existing hospital a was treated, but a state where sequelae remain is indicated by yellow or the like, and the nephritis is indicated by red or the like while still being treated. At this time, the kidney model may express a process of increasing or decreasing the medical information model according to the size development of the lesion. At this time, the visualization module 300 may adjust the color characteristics by interlocking the blood test results. An rgb transparency value may be determined and visualized by adding an algorithm that determines the color as a value returned by a function of using a blood creatinnine test result indicating a kidney function as a factor.

Then, the patient visits the hospital c again when the hepatitis occurs, and after the treatment is completed, information about the disease is transmitted from the medical institution H by the reception module 100, the text-type data 220 is collected by the data conversion module 200, and then the medical information model 312 is generated from the text-type data 220 by the visualization module 300 to complete the image-type data 310 for the hepatitis. The image-type data 310 for the hepatitis is stored by the storage-transmission module 500. Subsequently, the patient is hospitalized and discharged from a hospital d for treatment of fractures in the left leg. Nephritis is completely treated in the patient's personal terminal D, and the medical information model 312 for nephritis is removed from the basic model 311 by the visualization module 300. On the other hand, fracture lesions requiring observation may be displayed in green. As such, to express personal health as information received from the hospital, a lesion model and additional information related to the lesion model, that is, a name of a disease, such as stroke and hepatitis, a course of treatment, a drug during treating, blood tests associated with the corresponding disease, and various functional test results may be stored simultaneously as attributes.

FIG. 3 is a schematic diagram illustrating an embodiment of expressing symptoms of a patient. The patient makes an outpatient reservation for the pain generated in the left foot as a main symptom, and displays a painful part in the basic model 311 by using the display module 400 in the patient's personal terminal D. At the same time, the patient transmits his or her condition by adding additional information called “pain”. At this time, a time of occurrence, a cause of occurrence, and a place of occurrence may be transmitted as additional information. Meanwhile, the image-type data 310 drawn and received is stored in the remote storage 510. Then, when the patient visits the medical institution H, the information recorded in the personal health record generated by the health record system of the present invention is requested by the storage-transmission module 500, and the requested information may be shown to the medical staff in the electronic medical record system C of the hospital.

FIG. 17 shows a means for managing a plurality of patients in the medical institution H or the country. Patients with different lesions are represented as the image-type data 310 to provide better functions as compared with the conventional text-type data 220 in patient health management such as intensively managing the patient by checking the patient's disease condition at a glance and screening patients who may have problems or managing patients with similar diseases or patients having similar severity, and patients with diseases occurring in similar organs.

By the technical solution, according to the present invention, it is possible to simply and accurately organize personal medical information existing across several hospitals in a place. Such medical information may be visualized and displayed on a personal device for individuals without specialized knowledge. Further, it is possible to easily transmit symptom information to the medical institution H even when the patient does not clearly know the medical terminology in which the symptom information of the patient is recorded. Further, it is possible to provide a health record system capable of generating medical data 210 generated in each hospital in the form of a 2D or 3D model to convert the medical data into image-type data. This information may be structured as data that is more useful than previous technologies. In addition, the present invention may provide a health record system for standardizing text-type materials which are generated in each hospital to have different lengths while having ambiguous meanings. Further, it is possible to provide a health record system capable of having richer meanings by converting medical information recorded as the text-type data into the image-type data. In addition, the present invention may be used for chronic disease management, blood pressure nutrition, exercise management, etc. of each patient. In addition, the present invention enables collective management capable of managing diseases nationally by personalizing disease data. In addition, by using the system of the present invention, the user's disease data may be remotely transmitted to be efficiently used for customized consultation and treatment. In addition, by using the system of the present invention, it is possible to provide a national-level infrastructure capable of not only improving medical quality and efficiency but also contributing to the development of medical-related industries and economic development through this.

As described above, it will be understood to those by those skilled in the art that a technical configuration of the present invention can be easily executed in other detailed forms without changing the technical spirit or an essential feature thereof.

Therefore, the embodiments described as above are exemplary in all aspects and should be understood as not being restrictive and the scope of the present disclosure is represented by claims to be described below rather than the detailed description, and it is to be interpreted that the meaning and scope of the claims and all the changes or modified forms derived from the equivalents thereof come within the scope of the present invention.

### EXPLANATION OF REFERENCE NUMERALS AND SYMBOLS

| | |
|---|---|
| D. Terminal | H. Medical institution |
| C. Electronic medical record system | 100. Reception module |
| 200. Data conversion module | 210. Medical data |
| 220. Text-type data | 300. Visualization module |
| 310. Image-type data | 311. Basic model |
| 312. Medical information model | 320. Texture |
| 321. Round pattern | 322. Thin diagonal pattern |
| 323. Thick diagonal pattern | 324. Dotted diagonal pattern |
| 400. Display module | 500. Storage-transmission module |
| 510. Remote storage | |

1. A health record system comprising: a reception module configured to receive medical data including medical information generated in a medical institution;
a data conversion module configured to extract text-type data from the medical data;
a visualization module configured to generate image-type data by using the text-type data extracted by the data conversion module; and
a storage-transmission module configured to store and transmit the data generated by the reception module, the data conversion module, and the visualization module.

2. The health record system of claim 1, further comprising:
a display module configured to display the image-type data to a user.

3. The health record system of claim 1, wherein the visualization module generates the image-type data by combining one or more medical information models.

4. The health record system of claim 1, wherein the visualization module expresses the name of a disease, the severity of the disease, the chronicity, the degree of malignancy, various test results, functional test results, and data results extracted from a machine as any one or more of colors, brightness or transparency, patterns, and textures of the visualization data.

5. The health record system of claim 1, wherein the visualization module generates the image-type data by combining one or more medical information models.

6. The health record system of claim 1, wherein the image-type data generated by the visualization module is a predetermined 2D or 3D model.

7. The health record system of claim 1, wherein the data conversion module collects the medical data by receiving materials from any one or more of a portable file, a hospital, a cloud server, and a personal device.

8. The health record system of claim 1, wherein the visualization module uses an image extracted from the medical image or an anatomical pathology photograph as a texture.

9. The health record system of claim 1, wherein the visualization module uses one or more layers according to characteristics of the medical data.

10. The health record system of claim 1, wherein the visualization module further includes a medical information model expressing medical information which is not anatomically related inside and outside the body in the basic model.

11. The health record system of claim 1, wherein the visualization module combines multiple image-type data having different creating time points to express a change over time.

12. The health record system of claim 1, wherein the visualization module generates the image-type data automatically for the text-type data.

13. The health record system of claim 1, wherein a medical event having one or more diagnosis names or symptoms is managed as the image-type data.

14. The health record system of claim 2, wherein the display module executes another application or program of a device where the display module is implemented by using a graphic user interface event.

15. The health record system of claim 2, wherein the display module performs a search function by receiving a location and an area displayed by the user.

16. The health record system of claim 2, wherein the display module expresses a patient's condition by adding and removing the medical information model to and from the image-type data.
